(19) United States
(12) Patent Application Publication
(10) Pub. No.: US 2009/0082727 A1
(43) Pub. Date: Mar. 26, 2009

(54) Credit Card Sized Injection Device

(75) Inventors: Claus Schmidt Moeller, Fredensborg (DK); Thomas Dedenroth Miller, Bronshoj (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/631,571

(22) PCT Filed: Jun. 27, 2005
(86) PCT No.: PCT/EP2005/053001

§ 371 Date: Dec. 1, 2008

(30) Foreign Application Priority Data
Jul. 5, 2004 (EP) 04015740.6

(51) Int. Cl. A61M 5/34; A61M 5/20

(52) U.S. Cl. 604/132; 604/240

(57) ABSTRACT

A mechanical operated injection device which has the size of a credit card. The very compact size is obtained by using a flexible reservoir (90) containing the drug to be delivered and a suction pump (60, 70, 80) for pumping the liquid drug from the flexible reservoir to the body of the user. The pump is driven in cycles by a spring assembly which is energized by the user when setting the dose to be injected. The injection device is further connected to a needle assembly by a snap mechanism only requiring a 90 degree rotation of the injection needle assembly in order to release the injection needle assembly from the injection device.

### THE TECHNICAL FIELD OF THE INVENTION

The invention relates to an injection device for delivering a liquid medicament from a flexible reservoir to the human body. The injection device is in credit card size and has automatic injection of the set dose.

The invention further relates to a connection between an injection device and an injection needle assembly.

### DESCRIPTION OF RELATED ART

A prior art delivery device is disclosed in US 2002/007154. In this prior art delivery device the liquid medicament is contained in a glass cartridge. Normally 3 ml. of fluid medicament is stored in such glass cartridge. Further the delivery device comprises a piston rod which must have a length sufficient to press the entire content of the glass cartridge out through a conduit mounted on the distal end of the delivery device. As disclosed in US 2002/007154, the piston rod is bendable in order to shorten the over all length of the delivery device, this however adds to the width of the delivery device.

Further a compact, portable, pre-filled single use auto injector is disclosed in WO 03/099358.

### DESCRIPTION OF THE INVENTION

It is an object of the present invention to provide a delivery device from which set doses can be administered and which is even more compact than the hereto known devices.

It is a further object to provide a connection between a drug delivery device and a needle assembly which is simple in use.

### Claim 1

A very compact injection device can be obtained by providing an injection mechanism based on an expelling means that are driven in cycles such as a pump. In this way the traditional piston rod can be avoided and replaced by a flexible reservoir from which the drug is pumped. The user energized spring assembly for driving the expelling mechanism through a number of cycles can be provided in various forms but is preferably a spring loaded mechanism in which the spring is energized by the user e.g. by tightening the spring element.

### Claim 2

When the user energizes the spring means, the spring assembly or at least a part of the spring assembly is operated from an initial position to a set position thereby tightening the spring. Further during injection, the spring assembly or at least a part of it is moved from the set condition to the initial position thereby releasing the energy saved during setting of the dose.

### Claim 3

In order to release the energy stored, release means are provided which release means keeps the spring means in the set condition against the bias of the spring means. The energy stored is then released by actuating the release means. When released, the stored energy is used to drive the spring assembly from its set position to its initial position thereby driving the pump means through a number of cycles.

### Claim 4

By including a cam wheel with a number of cam elements which consecutively causes the actuation member to be driven through a number of cycles, the number of cycles can be indefinite only depending on the number of cam elements and the number of revolutions of the cam wheel.

### Claim 5-7

The pump means are preferably a piston actuated membrane pump which drives through the cycles of acquiring a quantity of liquid from the reservoir and expelling the same quantity. By using such pump the overall dimensions of the injection device can be diminished.

Such piston actuated membrane pump is disclosed in EP 1.525.873, which is hereby incorporated by reference.

### Claim 8

According to a further embodiment, the flexible reservoir containing the liquid drug can be made as a pre-filled and sealed flexible reservoir. Such flexible reservoir is preferably made from two foils which are connected in a pouch-like configuration and filled with a liquid drug.

An example of such flexible reservoir is disclosed in EP 1.525.873, which is hereby incorporated by reference.

### Claim 9

The pump means is equipped with an inlet means such as a conduit communicating with the reservoir and an outlet means communication with an injection needle assembly. In this way a fluid passage between the reservoir and the patient can be created through which fluid passage the liquid drug can be pumped.

### Claim 10

Further the injection device is of the type having what is known as dial-up/dial-down, which means a dose can be set i.e. by moving a member in a first direction where after the user if he regrets the set dose can diminish the setting by moving the same member in the opposite direction without the need for further handling and without the need for returning to a zero position.

When the user sets a dose by moving the dose setting means, the spring assembly or at least a part of the spring assembly is moved from an initial position to a set position. The movement of the spring assembly energizes the spring means. An example of this would be a spring that is tightened. The spring is retained in the tightened position by a release mean which could have the form of a push button interacting with the cocked spring. When a push button is activated, the spring is released and drives the drive assembly back to its initial position. During this backwards movement, the drive assembly or at least a part of the drive assembly operates the pump means, which in a preferred example is a cam mechanism that consecutively works the piston of the piston actuated membrane pump. The forth- and backwards movement of the membrane causes the pump to acquire drug from the flexible reservoir and subsequently expel the acquired drug through the injection needle connected to the drug delivery device.

### Claim 12-14

A injection needle assembly is preferably connected to the drug delivery device by a snap fastening mechanism. This mechanism is designed such that the injection needle assembly can be connected only by a pushing movement and released by rotating the injection needle assembly less than one full revolution.

The drug outlet on the injection device comprises a plurality of protrusion which is arranged in pairs. One pair is provided with detents which detents are arrested in recesses in the hub when the injection needle assembly is mounted on the injection device. In order to release the injection needle assembly, the hub is rotated to a position where the recesses are aligned with a pair of protrusions not carrying detents. The protrusion preferably forms a square-shaped opening into which the hub of injection needle assembly is pressed

### BRIEF DESCRIPTION OF THE DRAWINGS

The invention will be explained more fully below in connection with a preferred embodiment and with reference to the drawings in which:

FIG. 1 shows an exploded view of the injection device seen from the back.

FIG. 2 shows an exploded view of the spring connection.

FIG. 3 shows a view of the release button.

FIG. 4 shows an exploded view of the click wheel and its interfaces.

FIG. 5 shows an exploded view of the injection device seen from the front.

FIG. 6 shows an exploded view of the cyclic expelling mechanism.

FIG. 7 shows a view of the flexible reservoir and the holder.

FIG. 8 shows a cross sectional view of the injection device.

FIG. 9 shows a schematic view of the suction pump.

FIG. 10 shows a view of the injection needle assembly.

FIG. 11 shows a view of the container for the injection needle assembly.

FIG. 12 shows a view of the needle magazine.

FIG. 13 shows a view of the connection between the needle assembly and the connector.

FIG. 14 shows a view of the injection device with the injection needle assembly mounted in the connector.

FIG. 15 shows a view of the needle magazine used for rotating the needle assembly.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and left”, horizontal” and “vertical”, “clockwise and “counter clockwise” or similar relative expression are used, these only refer to the appended figures and not to an actual situation of use. The shown figures are schematic representations for which reason the configuration of the different structures as well as there relative dimensions are intended to serve illustrative purposes only.

Initially it may be convenient to define that the term “distal end” is meant to refer to the end of the injection device carrying the injection needle whereas the term “proximal end” is meant to refer to the opposite end pointing away from the injection needle.

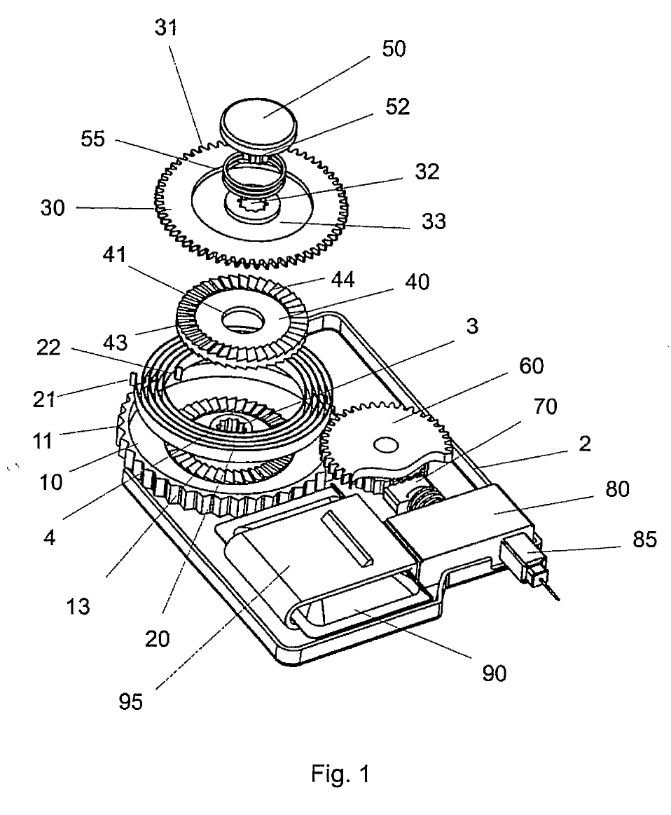

FIG. 1 shows an exploded view of the injection device 1 which comprises a housing 2 containing the dose setting and injection mechanism.

The dose and injection mechanism comprises a dose setting wheel 10 which is rotatable mounted in the housing 2 and accessible from outside the housing 2. On the periphery the dose setting wheel 10 is provided with a toothed surface 11 making it convenient to rotate the dose setting wheel 10 with the fingers. The dose setting wheel 10 is via a click wheel 40 coupled to an injection wheel 30 having gearing teeth 31 on its periphery. The click wheel 40 is sandwiched between the dose setting wheel 10 and the injection wheel 30.

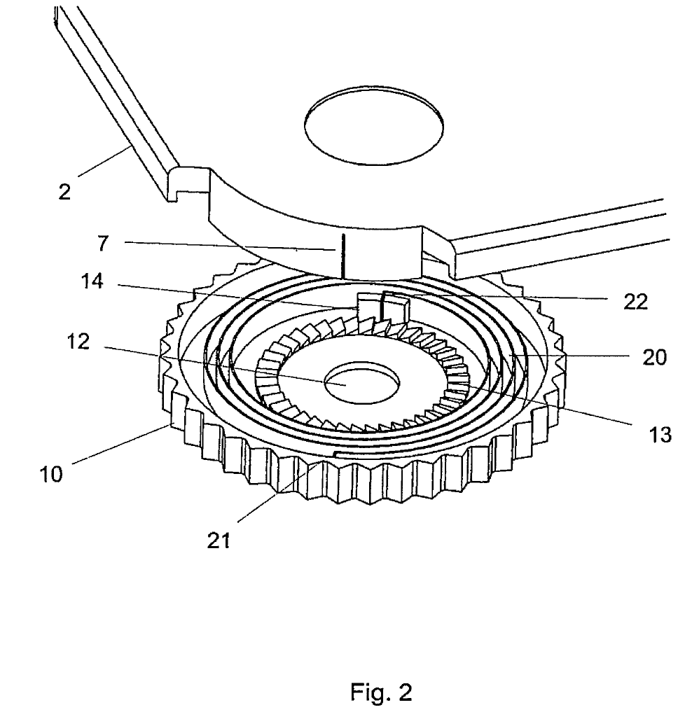

As disclosed in FIG. 2 a spring 20 is located in a cavity in the dose setting wheel 10. This spring 20 has a first end 21 secured in a slot 7 in the housing 2 and a second end 22 fastened to the dose setting wheel 10 that has an inwardly pointing protrusion 14 for holding the spring 20.

The housing 2 is on an interior surface provided with a circular protrusion 3 which functions as a bearing for the dose setting and injection mechanism. The injection wheel 30 is rotatable located with its centre portion on the top of protrusion 3. The click wheel 40 is rotatable located with its centre opening 41 surrounding the protrusion 3, and the dose setting wheel 10 is also rotatable mounted with its central opening 12 surrounding the protrusion 3.

The click wheel 40 has a first rim of teeth 43 provided on a first side and a second rim of teeth 44 provided on a second side thereof. The click wheel 40 is sandwiched between the dose setting wheel 10 and the injection wheel 30 with the first side pointing toward the dose setting wheel 10 and the second side pointing toward the injection wheel 30.

The first rim of teeth 43 interacts with an opposite located third rim 13 of teeth on the dose setting wheel 10, and the second rim of teeth 44 interacts with an opposite fourth rim of teeth 34 located on the injection wheel 30.

The first rim of teeth 43 and the third rim of teeth 13 are pointed in different directions such that they are locked to each other in one rotational direction but rotatable relatively to each other in the opposite direction. This is also the case for the second rim of teeth 44 and the fourth rim of teeth 34.

The protrusion 3 on the interior of the housing 1 has an internal cavity 4 with a non-circular shape. This non-circular shaped cavity 4 supports a release button 50 such that the release button 50 is inrotatable connected to the protrusion 3 and thereby to the housing 2.

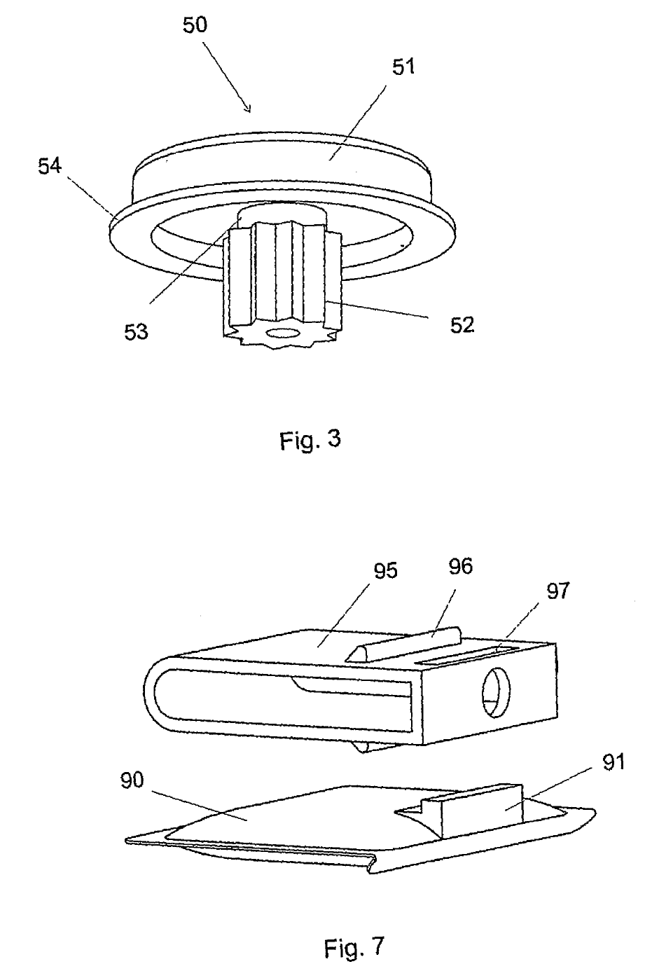

The release button 50 shown in details in FIG. 3 comprises a head 51 which is located outside the housing 2 and a centrally located rod 52 which extends into the non-circular cavity 4 in the housing 2. The rod 52 has a non-circular shape fitting the non-circular cavity 4 in the protrusion 3. At the junction between the head 51 and the rod 52 a circular indentation 53 is present. This circular indentation 53 has a diameter smaller than the diameter defined by the non-circular rod 52.

The injection wheel 30 has a centrally located hole 32 with a non-circular shape fitting the non-circular shape of the protrusion 3 on the housing 2 such that the injection wheel 30 is inrotatable mounted to the release button 50 and to the housing 2.

The release button 50 is secured in the housing 2 such that it can be shifted between a first position and a second position transversely to the housing 2 as best seen in FIG. 8. The release button 50 could e.g. be secured in the housing 2 by a flange 54 located on the release button but inside the boundaries of the housing 2. A spring 55 is provided between the head 51 of the release button 50 and the injection wheel 30 urging the injection wheel 30 against the click wheel 40 and the dose setting wheel 10.

In the first position, the spring 55 located between head 51 of the release button 50 and the injection wheel 30 urges the release button 50 away from the injection wheel 30 and vice versa. In this first position the non-circular shaped rod 52 locks the injection wheel 30 inrotatable to the housing 2.

In the second position, the release button 50 is pressed towards the housing 2 against the bias of the spring 55. In this second position, the circular indentation 53 is moved into the centrally located hole 32 in the injection wheel 30 such that the injection wheel 30 is free to rotate relatively to the release button 50 and the housing 2

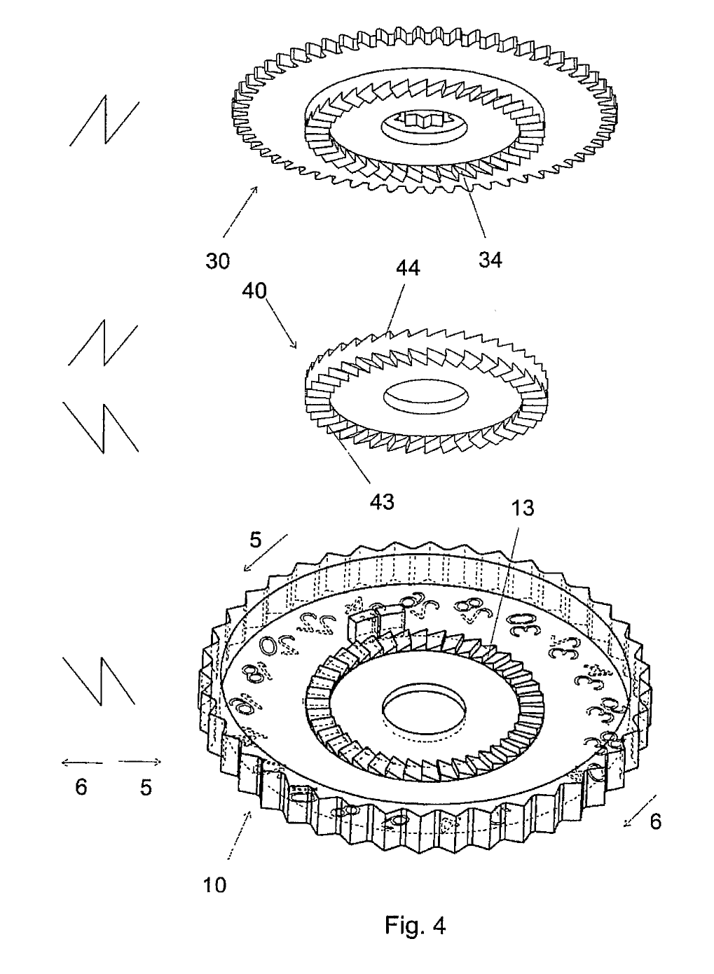

Now referring to FIG. 4, if a user wants to set a dose, the user rotates the dose setting wheel 10 in a counter clockwise direction (when viewed from the back as in FIG. 1) as indicated by the arrow 5, this movement tightens the spring 20. It is apparent that if the user moves the dose setting wheel 10 to far in the counter clockwise direction i.e. sets to large a dose, the dose setting wheel 10 can instantly be rotated in the clockwise direction thereby reducing the size of the set dose.

As the dose setting wheel 10 is rotated in the counter clockwise direction, the third rim of teeth 13 will ride over the first rim of teeth 43 on the click wheel 40 making an audible sound preferably indicating the number of doses being set. Since the injection wheel 30 is locked by the release button 50, the click wheel 40 is locked against rotation in the counter clockwise direction due to the interaction between the second rim of teeth 44 and the fourth rim of teeth 34. When the dose is reduced by rotating the dose setting wheel 10 in the clockwise direction, the click wheel 40 will rotate simultaneously with the dose setting wheel 10 due to the interaction between the first rim of teeth 43 and the third rim of teeth 43. At the same time the second rim of teeth 44 will ride over the fourth rim of teeth 34 on the injection wheel 30.

The force of the spring 55 is balanced such that the dose setting wheel 10 will remain in its set position when the finger of the user is removed from the toothed surface 11 i.e. the dose setting wheel 10 is held by the force urging the second rim of teeth 44 and the fourth rim of teeth 34 together.

In order to inject the set dose, the user activates the release button 50 shifting it to its second position. In this position the injection wheel 30 is free to rotate in the indentation 53.

The spring 20 will then return to its initial position pulling the dose setting wheel 10 in the clockwise direction as indicated by the arrow 6. This will force the click wheel 40 to rotate simultaneously due to the interaction between the first rim of teeth 43 and the third rim of teeth 3. Since the injection wheel 30 is pressed against the click wheel 40 by the flange 54 on the release button 50 which fits into the cavity 33 in the injection wheel 30, the second rim of teeth 44 will be in engagement with the fourth rim of teeth 34 on the injection wheel 30 forcing the injection wheel 30 to rotate in the clockwise direction. The distance between the flange 54 and the cavity 33 is such that the fourth rim of teeth 34 can not disengage the second rim of teeth 44 when the release button 50 is in its second position

During the clockwise rotation of the injection wheel 30, the gearing teeth 31 on the periphery will force a hammer wheel 60 to rotate.

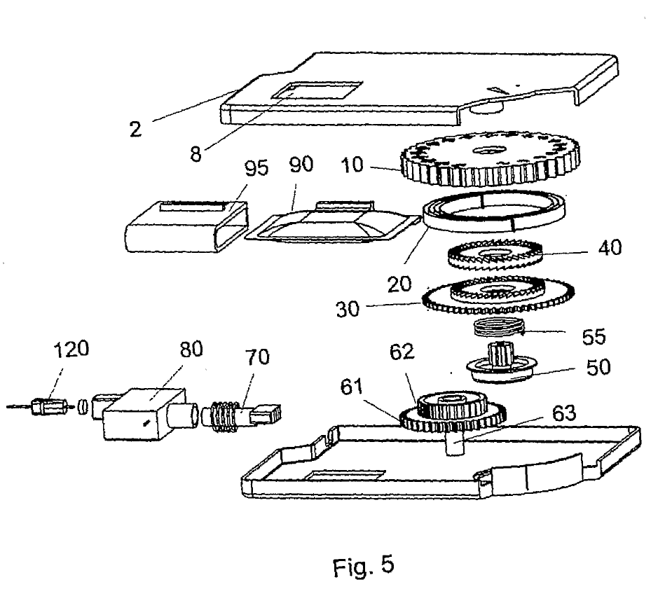

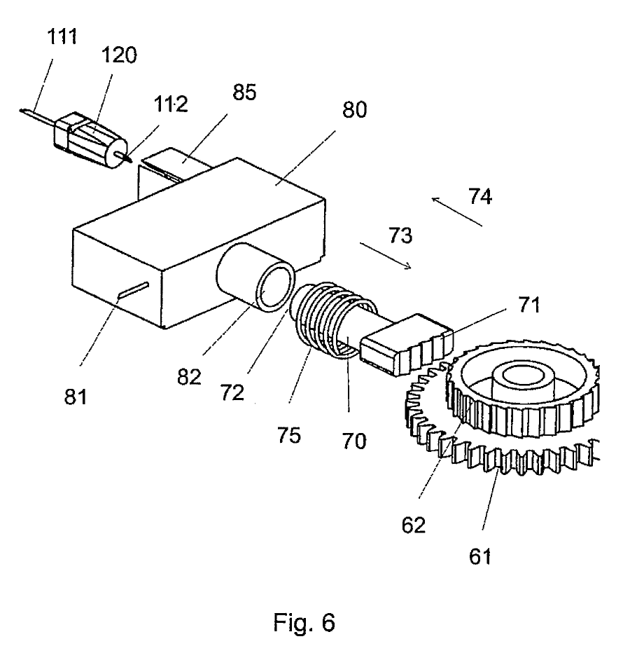

The hammer wheel 60 depictured in FIG. 5 and FIG. 6 comprises a first toothed periphery 61 engaging the injection wheel 30 and a second toothed periphery 62 engaging a hammer 70. The first toothed periphery 61 and the second toothed periphery 62 are connected or integral such that they rotate unison and are rotatable connected to the housing 2 through a common axis 63.

The toothed engagement between the second toothed periphery 62 on the hammer wheel 60 and the proximal toothed surface 71 on the hammer 70 transforms the rotational movement of the hammer wheel 60 to a linear movement of the hammer 70. As the hammer wheel 60 rotates, the toothed surface 71 and the spring 75 will alternate the hammer 70 forward and backwards as indicated by the arrows 73 and 74.

A not shown connection between the proximal toothed surface 71 on the hammer 70 and the hammer wheel 60 will dampen the interface such that the hammer movement will follow the toothed engagement. The connection could e.g. be an extra wheel interfaced between the hammer wheel 60 and the hammer 70 or a similar mechanism limiting the distance that the toothed surface 71 can be moved away from second toothed periphery 62 of the hammer wheel 60 in order to secure engagement.

The distal end 72 of the hammer 70 is inserted into the tube 82 such that the alternating movement of the hammer 70 is transformed to the pump unit 80.

The pump unit 80 further comprises a conduit 81 and a drug outlet 85 connectable to an injection needle. The conduit 81 is connected to a flexible reservoir 90 containing the liquid medicament to be injected.

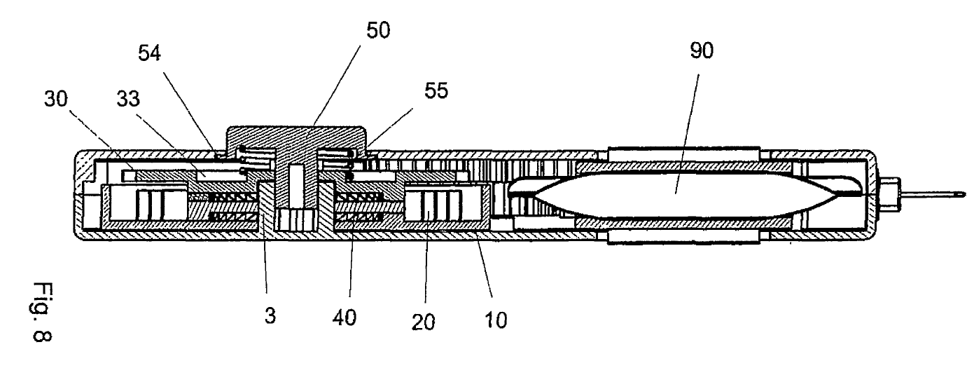

The flexible reservoir 90 shown in FIG. 7 comprises a bag-like construction made from a suitable polymer which bag contains the liquid medicament. The flexible reservoir 90 is provided with an entrance 91 through which the conduit 81 can be inserted into the interior of the flexible reservoir 90. The entrance 91 could e.g. be connected to the flexible reservoir 90 or made as an integral part of the flexible reservoir 90.

The flexible reservoir 90 is positioned in a holder 95 which is movable connected in the housing 2. The holder 95 has a pair of finger grips 96 which are accessible for a user through a number of openings 8 in the housing and an opening 97 for supporting the entrance 91. The holder 95 is guided in the opening 8 by the abutment of the end surfaces of the finger grips 96 against the side of the opening 8.

When a user wants to make the injection device 1 ready for injection, he slides the holder 95 with the reservoir 90 towards the conduit 81 such that the conduit 81 breaks through the entrance 91 and creates a fluid passage from the interior of the flexible reservoir 90 to the pump unit 80.

Thereafter he attaches a needle assembly 100 to the drug outlet 85 such that the back end 112 of the needle cannula 110 penetrates into the pump unit 80. When inserting the fore end 111 of the needle cannula 110 into the subcutaneous layer of the body of the user, a fluid passage is created from the reservoir 90 to the subcutaneous layer. The amount of fluid passing through this fluid passage is controlled by the pump unit 80.

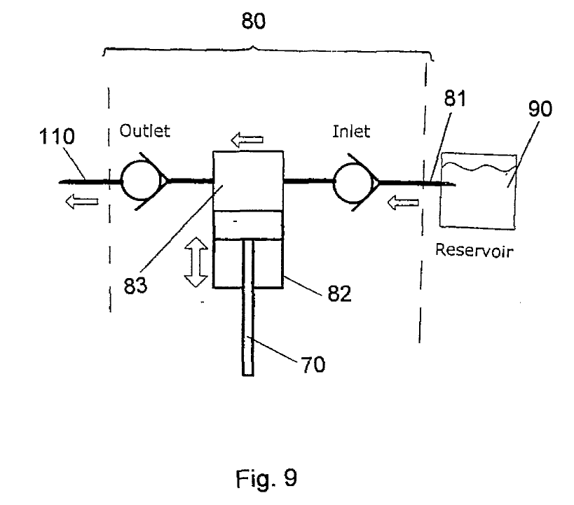

FIG. 9 is a schematic view of interior of the pump unit 80. The inlet side of the pump is connected to the reservoir 90 through the conduit 81 and the needle cannula 110 is connected to the outlet side. When the hammer 70 is linearly alternated, the suction chamber 83 is initially filled from the reservoir 90 as the hammer 70 moves out of the suction chamber 83, and the liquid is expelled through the needle cannula 110 when the hammer 70 is moved into the suction chamber 83.

The user sets the dosage by rotating the dose setting wheel 10 in the clockwise direction thereby tightened the spring 20. To release the set dose the user activates the release button 50 thereby making it possible for the spring 20 to drive the dose setting wheel 10 back to its initial position bringing the injection wheel 30 with it. This rotation of the injection wheel forces the hammer wheel 60 to rotate. The rotation of the hammer wheel 60 is translated into an alternating linear movement of the hammer 70, which movement operates the pump unit 80 to suck liquid drug from the reservoir 90 and to deliver the sucked drug to the needle cannula 110.

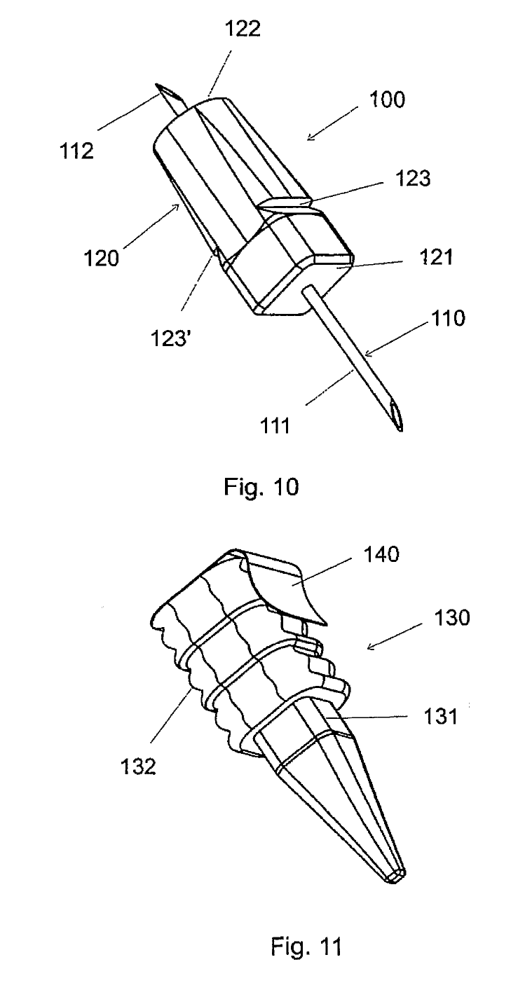

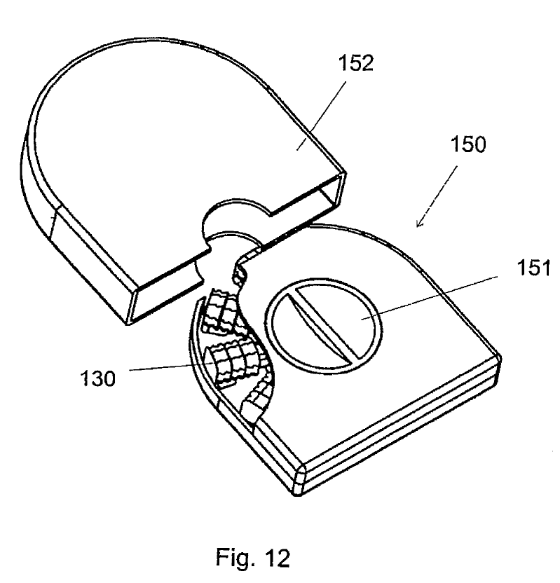

As can be seen from FIG. 10, the needle assembly 100 comprises a needle cannula 110 mounted in a hub 120. The needle cannula 110 is connected such that a fore end 111 points in the distal direction and a back end 112 points in the proximal direction. The individual needle assembly 100 is packed in a container 130 which is sealed by a peal foil 140 as shown in FIG. 11. A plurality of such needles assemblies 100 can be stored in a needle magazine 150.

As can be seen in FIG. 12, the needle magazine 150 is provided with a dial 151 by which the individual needle assemblies 100 can be brought into a loading position. Further the needle magazine 150 is provided with a cover 152 for protecting the needle assemblies 100 during transportation.

The hub 120 carrying the needle cannula 110 has a square cross section at the distal end 121 and a circular cross section at the proximal end 122. Between the distal end 121 and the proximal end 122, the cross section transforms from a square shape to a circular shape. Towards the distal end 121, the hub 120 is provided with two slots or recesses 123, 123′. These slots 123, 123′ is cut or moulded in the hub 120 at the junction were the square cross section transforms into the circular cross section. The slots 123, 123′ are located opposite each other.

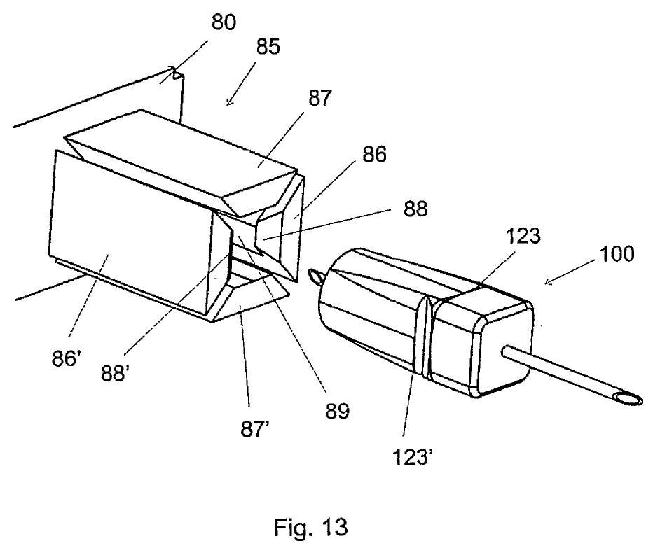

The drug outlet 85 shown in details in FIG. 13 comprises four protrusions 86, 86′, 87, 87′ separate by an equal number of slots such that each protrusion 86, 86′, 87, 87′ is flexible and can bend axially. The four protrusions 86, 86′, 87, 87′ is preferably integral with the housing of the pump unit 80 and forms together a square-shaped opening 89 allowing access to the pump unit 80. Two opposite located protrusions 86, 86′ has on the most distal end an inwardly pointing detent 88, 88′, while the two other protrusions 87, 87′ is without such detent.

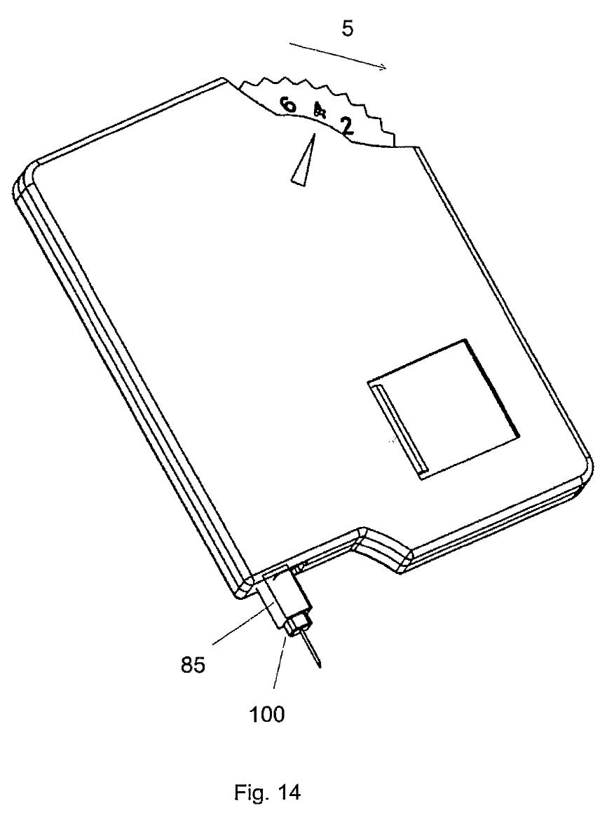

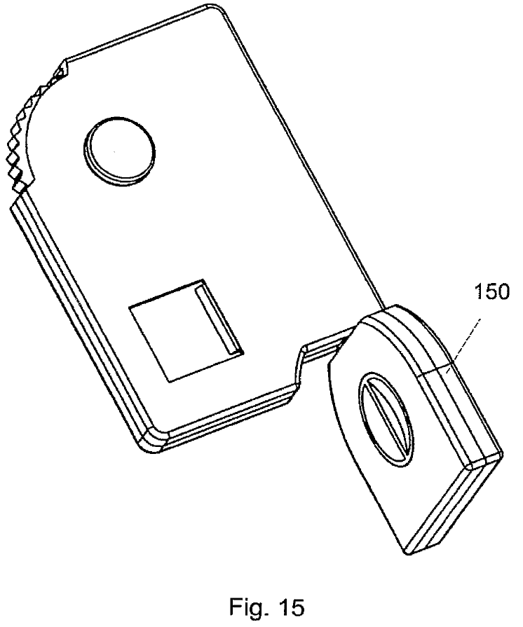

When the needle assembly 100 is pressed into the opening 89 of the drug outlet 85, the two detents 88 will enter into the two slots 123 and lock the needle assembly 100 in the correct position as disclosed in FIG. 14.

In order to release the needle assembly 100, the user only has to rotate the needle assembly 100 approximately 90 degrees such that the slots 123 are aligned with the protrusion without detents 87, 87′. The needle magazine 150 can be used as a tool for performing this rotation as disclosed in FIG. 15.

In order to allow this operation, the container 130 containing the needle assembly 100 is provided with a square portion 131 which fits the square part of the needle hub 120. The proximal part of the container 130 is preferably formed as an accordion allowing it to be folded when it interacts with the drug outlet 85, the container 130 could also be made large enough to fit outside the protrusions 86, 86′.

Some preferred embodiments have been shown in the foregoing, but it should be stressed that the invention is not limited to these, but may be embodied in other ways within the subject matter defined in the following claims.

1. An injection device (1), comprising:
a flexible reservoir (90) adapted to contain an amount of a liquid drug,
a drug outlet (85),
pump means (60, 70, 80) adapted to perform one or more cycles of pumping a pre-determined amount of drug contained in the flexible reservoir (90) through the drug outlet (85),
dose setting means (10) for selectable setting a dose of drug to be pumped, and
a user energized spring assembly (20, 30, 40) operatively coupled to the dose setting means (10) for driving the pump means (60, 70, 80) through a number of cycles in accordance with the set dose.

2. An injection device as defined in claim 1, wherein the spring assembly (20, 30, 40) comprises:
spring means (20) operatively coupled to the dose setting means (10) such that the spring means (20) is energized when the drive assembly (20, 30, 40) is operated from an initial condition to a set condition corresponding to a set dose,
wherein the spring means (20) is adapted to drive the spring assembly (20, 30, 40) from its set condition to its initial condition thereby causing the pump means (60, 70, 80) to be driven through a number of cycles in accordance with the set dose.

3. An injection device as defined in claim 2, further comprising:
release means (50) adapted to retain the spring assembly (20, 30, 40) in the set condition against the bias of the spring means (20),
whereby actuation of the release means (50)allows the spring means (20) to drive the spring assembly (20, 30, 40) from its set condition to its initial condition.

4. An injection device as defined in claim 2, wherein the pump means (60, 70, 80) comprises a cam wheel (60) with a plurality of cam members (62) and an actuation member (70) operatively coupled to the cam wheel (60) such that rotation of the cam wheel (60) in accordance with the set dose consecutively causes the cam members (62) to drive the actuation member (70) through a number of cycles.

5. An injection device as defined in claim 1, wherein the pump means (60, 70, 80) comprises a suction pump (80) adapted to pump drug from the flexible reservoir (90) to the drug outlet (85).

6. An injection device as defined in claim 5, wherein the suction pump (80) when actuated cycles through a mode of acquisition of a quantity of the liquid drug from the flexible reservoir (90) and a subsequent mode of expulsion of the quantity of liquid drug acquired from the flexible reservoir (90).

7. An injection device as defined in claim 6, wherein the suction pump (80) is in the form of a piston actuated membrane pump (80) comprising an inlet valve associated with the flexible reservoir (90) and an outlet valve associated with the drug outlet (85).

8. An injection device as defined in claim 7, wherein the flexible reservoir (90) is a prefilled and sealed flexible reservoir (90).

9. An injection device as defined in claim 1, wherein the pump means (60, 70, 80) comprises an inlet (81) adapted to be arranged in fluid communication with the flexible reservoir (90), and an outlet (85) adapted to be arranged in fluid communication with an injection needle assembly (100), thereby allowing the injection needle assembly (100) to be arranged in fluid communication with the interior of the flexible reservoir (90).

10. An injection device as defined in claim 9, wherein the dose setting means (10) allows a first dose to be selected and subsequently adjusted to a second lower dose without actuation of the spring means (20).

11. A connection between an injection needle assembly (100) and an injection device (1), which comprises in combination:
An injection needle assembly (100) comprising a needle hub (120) provided with a plurality of recesses (123),
An injection device (1) including a drug outlet (85) comprising a plurality of protrusions (86, 87), wherein at least one of the protrusions (86, 87) has a detent (88) which interacts with at least one of the recesses (123) for securing the needle assembly (100) in a use position.

12. A connection between an injection needle assembly (100) and an injection device (1) as defined in claim 11, wherein the plurality of protrusions (86, 87) forms an opening (89) into which the needle hub (120) fits and wherein at least one detent (88) points towards the opening (89).

13. A connection between an injection needle assembly (100) and an injection device (1) as defined in claim 12, wherein the opening (89) is square-shaped and formed between four protrusions (86, 86′, 87, 87′).

14. A connection between an injection needle assembly (100) and an injection device (1) as defined in claim 13, wherein the detents (88, 88′) are provided on the most distal inwardly pointing side of two opposite located protrusions (86, 86′) and wherein the recesses (123, 123′) are provided on opposite sides of the needle hub (120).
